(19) United States
(12) Patent Application Publication
(10) Pub. No.: US 2026/0207123 A1
(43) Pub. Date: Jul. 23, 2026

(54) A PREGNANCY TEST APPARATUS WITH AFFIRMATION DISPLAY AND METHOD

(71) Applicant: Bridger YATES, Logan, UT (US)

(72) Inventor: Bridger YATES, Logan, UT (US)

(21) Appl. No.: 19/142,427

(22) PCT Filed: Jun. 26, 2024
(86) PCT No.: PCT/US2024/035594

§ 371 Date: Jun. 23, 2025

Related U.S. Application Data
(60) Provisional application No. 63/524,899, filed on Jul. 4, 2023.

(51) Int. Cl. A61B 5/00

(52) U.S. Cl. CPC A61B 5/4343; A61B 5/742

(57) ABSTRACT

A pregnancy test apparatus with a display configured to show a positive affirmation to a user in the event of a negative test result that indicates that the user is not pregnant. The positive affirmation may include messages urging the user to maintain a positive emotional state, that she is not alone, can succeed, and to continue trying. Also, a pregnancy test method that comprises the steps of providing a pregnancy test apparatus with the above features and displaying the positive affirmation in the event of a negative test result that indicates that the user is not pregnant.

### TECHNICAL FIELD AND INDUSTRIAL APPLICABILITY OF THE INVENTION

The invention relates to human pregnancy tests and human pregnancy testing methods and pregnancy test displays.

### BACKGROUND

Family planning has a long tradition and is arguably one of the most meaningful aspects of the human experience. For those who choose to have children, the need to detect and monitor pregnancy is paramount. Two common methods for testing pregnancy in women are, first, testing for the female pregnancy hormone (human chorionic gonadotropin (hCG)) in blood or urine using a pregnancy test kit, second, and scanning with ultrasonography. Pregnancy test kits that may be purchased over-the-counter are more common among these methods, simply because they are easily accessible, relatively inexpensive, self-administered, and private.

When people are trying to get pregnant, hopes and expectations are high. Any number of obstacles such as delay, partner separation or absence, infertility, and other health issues, can challenge those hopes and expectations. It is well recognized that such obstacles can include societal repercussions, personal suffering, and adverse psychological effects and emotional consequences such as anger, depression, anxiety, marital problems, and feelings of worthlessness.

It is common to experience many pregnancy tests in the face of these efforts, obstacles, and challenges. Standard and anticipated indicators and/or results displays from positive pregnancy tests may include “double bars” or, in the case of digital displays “Pregnant.” In contrast, negative pregnancy tests may show a “single bar” or simply “Not Pregnant.” These kind of standard indicators of negative pregnancy tests can seem cold, indifferent, and uncaring for those experiencing obstacles and challenges in getting pregnant. Accordingly, there is a great need to provide support and motivation to those who are trying to get pregnant, but who are encountering obstacles and challenges along the way.

What is needed is a new type of pregnancy test and method that includes motivating messaging and positive affirmation in connection with negative or disappointing results.

### SUMMARY OF THE INVENTION

In accordance with the above, the invention includes a pregnancy test with display configured to share an affirmation with any negative test result and a method relating to the same.

### BRIEF DESCRIPTION OF THE DRAWINGS

To further clarify the above and other aspects of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The drawings may not be drawn to scale. The invention will be described and explained with additional specificity and detail through the use of the accompanying drawings in which:

FIG. 1 is a plan view of a pregnancy test in one embodiment.

FIG. 2 is an exploded view of a pregnancy test in one embodiment.

FIG. 3 is a plan view of various affirmations in one embodiment.

FIG. 4 is a block diagram representing a method of using the pregnancy test in one embodiment.

### DETAILED DESCRIPTION OF EMBODIMENTS AND MODES FOR CARRYING OUT THE INVENTION

The present invention in its various embodiments, some of which are depicted in the figures herein, is a pregnancy test with affirmation display and method. The illustrated embodiments are shown for exemplary purposes only.

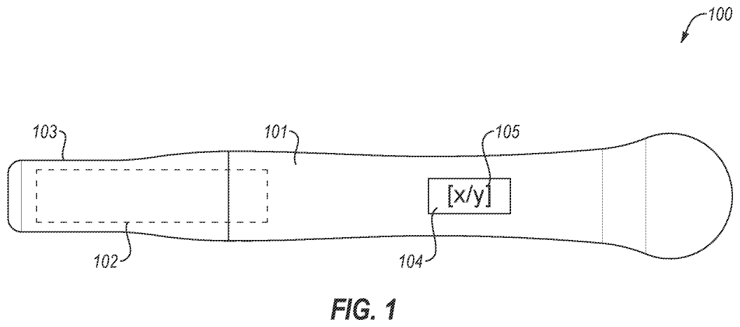

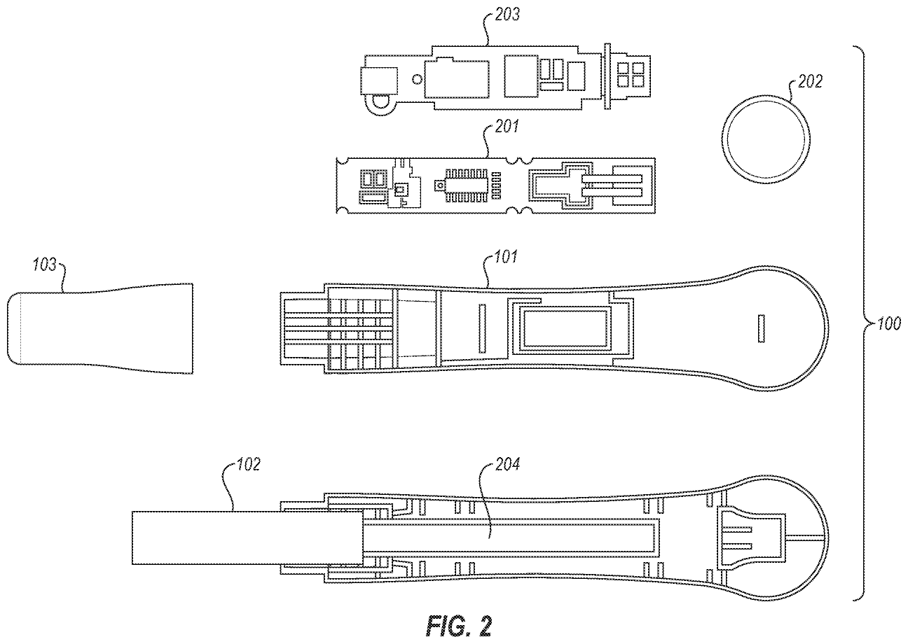

Referring now to FIGS. 1-2 , an exemplary pregnancy test apparatus is shown. For illustrative purposes, the pregnancy test apparatus 100 is depicted as an over-the-counter, female pregnancy hormone (human chorionic gonadotropin [hCG]), urine testing pregnancy test kit with a digital display. However, other types of pregnancy tests may be used without departing from the purposes and scope of the invention. Pregnancy test 100 may include a plastic body 101 with a display 104 and a sample testing portion or strip 102 with a lid or cover 103. Display 104 may be configured to show indicators for status and test results (e.g., X/Y) 105. Plastic body 101 may form a housing (with or without additional plastic elements, e.g., 203) for other components of the pregnancy test 100. Other components may include: a circuit board 201 having memory and processor for various modes of operation, including for generating modes and indicators on the display 104; a battery for powering the circuit board 202; and an HCG test strip 204 connected to an absorbent tip of the testing portion 102.

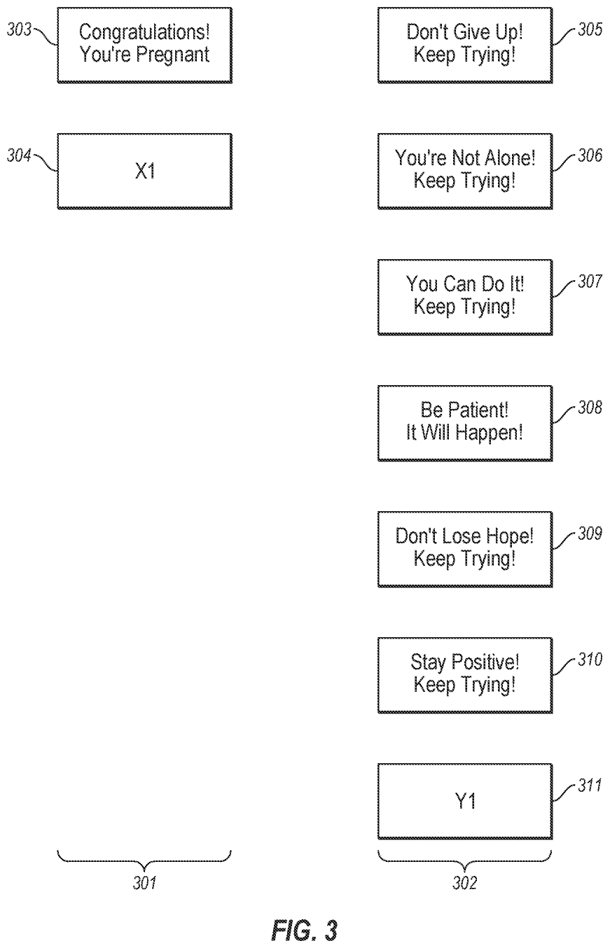

Referring now to FIG. 3, the display 104 is configured to show two categories of test results with test result indicators, among other status and function indicators: (1) a first category 301 indicating a positive test result and/or user pregnancy; and (2) a second category 302 indicating a negative test result and/or user non-pregnancy. Indicators for either the first or the second category may be icons, emoticons, symbols, words, a phrase, or phrases. In the preferred embodiment, indicators of the first category—shown on the display 104 in connection with a positive test result—may be of various standard types of congratulatory words and/or phrases, including the words “Congratulations! You're Pregnant” 303 and the like (X1, 304).

Indicators of the second category 302 may also be of various types of positive affirmation words and/or phrases including one or more word positive affirmations—shown on the display 104 in connection with a negative result—including: “Don't Give Up! Keep Trying!” 305; “You're Not Alone! Keep Trying!” 306; “You Can Do It! Keep Trying!” 307; “Be Patient! It Will Happen!” 308; “Don't Lose Hope! Keep Trying!” 309; “Stay Positive! Keep Trying!”310; and the like (Y1, 311). In the preferred embodiment, the positive affirmation may be and/or is specifically generated or shown on the display 104 one or more of prior to, concurrently with, and following a negative test result. In certain embodiments, the display 104 is configured to only show the positive affirmation one or more of prior to, concurrently with, and following a negative test result and/or the display of a negative test result.

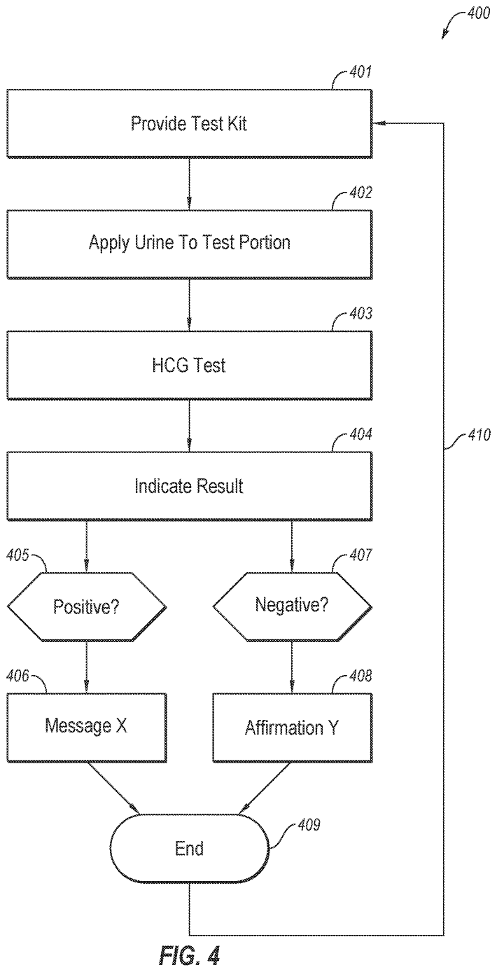

Referring now to FIG. 4, a method 400 relating to the pregnancy test is shown 100. First, a test kit with the above general features and/or configurations is provided 401, particularly with one or more positive affirmation indicators on the display that accompany any negative result. The person desiring to perform a pregnancy test then applies urine to the test portion 402, in the standard manner. An HCG (or other applicable) test is then conducted by the test kit 403. When the HCG (or other applicable) test is finished, the test kit translates the test result to display and/or indicate the result (pregnant vs. not pregnant) 404.

If the test shows a positive result 405, a suitable positive result affirmation is displayed 406 on the display. In contrast, if the test shows a negative result 406, a negative result is shown with affirmation (e.g., 305-311) on the display 408. The steps of the method may stop 409 and/or be repeated as necessary 410. As set forth above, the positive affirmation may be shown one or more of prior to, concurrently with, and following a negative test result.

Structured as set forth above, the pregnancy test with affirmation display and method convey a number of functional advantages and improvements. First, and foremost, the pregnancy test and method provide motivating messages in connection with negative test results to provide support and motivation to users who are trying to get pregnant, but who are encountering obstacles and challenges along the way.

The present invention may be embodied in other specific forms without departing from its spirit or essential characteristics. For example, displays may be analog or of any other type than digital. Moreover, the exact type of pregnancy test—including substrate tested or tests performed—may vary. Further, any number of different and/or specific affirmations, positive messages, words, or symbols may be incorporated. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

I claim:

1. A pregnancy test apparatus with a display configured to show a positive affirmation to a user one or more of prior to, concurrently with, and following a negative test result that indicates that the user is not pregnant.

2. The pregnancy test apparatus of claim 1 wherein the positive affirmation is comprised of one or more of an icon, emoticon, symbol, word, phrase, and phrases.

3. The pregnancy test apparatus of claim 1 wherein the positive affirmation is comprised of one or more words.

4. The pregnancy test of claim 3 wherein the positive affirmation is one or more of a message of words to the user informing the user: to continue efforts to become pregnant; that the user is not alone in attempting to become pregnant; to maintain a positive emotional state; and that the user can succeed in becoming pregnant.

5. The pregnancy test apparatus of claim 3 wherein the positive affirmation is generally one or more of the following phrases: Don't Give Up, Keep Trying, You're Not Alone, You Can Do It, Be Patient, Don't Lose Hope, and Stay Positive.

6. The pregnancy test apparatus of claim 5 wherein the display is configured to only show the positive affirmation one or more of prior to, concurrently with, and following the negative test result that indicates that the user is not pregnant.

7. The pregnancy test apparatus of claim 1 wherein the positive affirmation is substantially one of or more of the following phrases: Don't Give Up, Keep Trying, You're Not Alone, You Can Do It, Be Patient, It Will Happen, Don't Lose Hope, and Stay Positive.

8. The pregnancy test apparatus of claim 1 wherein the positive affirmation consists of one or more of the following phrases: Don't Give Up!, Keep Trying!, You're Not Alone!, You Can Do It!, Be Patient!, It Will Happen!, Don't Lose Hope!, and Stay Positive !.

9. The pregnancy test apparatus of claim 8 wherein the display is configured to only show the positive affirmation one or more of prior to, concurrently with, and following the negative test result that indicates that the user is not pregnant.

10. A pregnancy test method comprising the steps of:
providing a pregnancy test apparatus with a display configured to show a positive affirmation to a user one or more of prior to, concurrently with, and following a negative test result that indicates that the user is not pregnant; and
displaying the positive affirmation on the pregnancy test apparatus to the user one or more of prior to, concurrently with, and following the negative test result that indicates that the user is not pregnant.

11. The pregnancy test method of claim 10 wherein the positive affirmation is comprised of one or more of an icon, emoticon, symbol, word, phrase, and phrases.

12. The pregnancy test method of claim 11 wherein the positive affirmation is comprised of one or more words.

13. The pregnancy test method of claim 12 wherein the positive affirmation is one or more of a message of words to the user informing the user: to continue efforts to become pregnant; that the user is not alone in attempting to become pregnant; to maintain a positive emotional state; and that the user can succeed in becoming pregnant.

14. The method of claim 12 wherein the positive affirmation is generally one or more of the following phrases: Don't Give Up, Keep Trying, You're Not Alone, You Can Do It, Be Patient, Don't Lose Hope, and Stay Positive.

15. The method of claim 12 wherein the positive affirmation consists of one or more of the following phrases: Don't Give Up!, Keep Trying!, You're Not Alone!, You Can Do It!, Be Patient!, It Will Happen!, Don't Lose Hope!, and Stay Positive !.
